(19) United States
(12) Patent Application Publication
(10) Pub. No.: US 2010/0124760 A1
(43) Pub. Date: May 20, 2010

(54) Zinc Test Strip and Method for the Detection of Semen

(75) Inventor: Stephen Patrick Ashburn, Fort Meade, SD (US)

Correspondence Address: STEPHEN P. ASHBURN, P.O. Box 300, Fort Meade, SD, 57741, US

(73) Assignee: Stephen Patrick Ashburn, Fort Meade, SD (US)

(21) Appl. No.: 12/271,722

(22) Filed: Nov. 14, 2008

(51) Int. Cl. C12Q 1/02

(52) U.S. Cl. 435/29

(57) ABSTRACT

The present invention relates to a forensic test strip and method for the detection of semen. This strip is comprised of a paper element coated with reagents which react to the presence of zinc, a component of semen, and is affixed to a plastic backing to facilitate testing items without touching the highly sensitive coated portion of the strip. This assembly is exposed to a source of semen, such as a suspicious stain on a garment. A positive test is characterized by a bright pink color, which easily can be seen against the light yellow background. A positive test provides presumptive evidence of semen.

### FIELD OF THE INVENTION

The present invention relates to forensic chemistry. In particular, it relates to an analytical test and method thereof for the detection of semen on fabrics and other surfaces.

### BACKGROUND OF THE INVENTION

Conservative statistics indicate that about 14% of women and 22% of men have had affairs sometime in their marriage [Laumann et al. “The Social Organization of Sexuality: Sexual Practices in the United States”; University of Chicago Press: Chicago, page 216, 1994]. According to a recent study by the Centers for Disease Control, about 4% of both married men and women had more than one sexual partner in the previous twelve months [Mosher et al. “Sexual Behavior and Selected Health Measures: Men and Women 15-44 Years of Age, United States, 2002,” Advance Data, 362, page 10, 2005]. This figure rises to 15% in the case of unmarried couples cohabiting. These data indicate that infidelity is a significant problem in the United States, and there exists a need to objectively test spouses for sexual activity. For women, one such test is for the presence of semen.

When a man has sexual intercourse with a woman, semen is deposited into the woman's vagina. Immediately after intercourse, most of the semen flows back out, but some is retained in the vagina and slowly is discharged over a period of several days [Hooft et al, Am. J. Forensic Med. Pathol., 18, pages 45-49, 1997]. Semen has over 900 identified proteins [Pilch et al, Genome Biology, 7(R40), 2006] among which are semenogelin I and II (gel-forming proteins produced by the seminal vesicles), prostate-specific antigen (PSA, a protease which breaks down semenogelin), and acid phosphatase (which breaks down spermatozoa cell membranes) [Tanaka et al, FEBS Lett., 571, pages 197-204, 2004]. These proteins can be identified by immunochromatographic assay, which forms the principle of the PSA and semenogelin tests. Acid phosphatase can be detected by the classic test first reported by Babson et al in Am.J.Clin.Pathol., 32, pages 88-91 (1959), which forms the principle of the AP test. This test relies on the catalytic hydrolysis of 1-naphthyl phosphate to form 1-naphthol, which in turn reacts with an aryl diazonium salt, forming an intensely colored azo dyestuff. In addition to proteins, semen also has unusually high concentrations of zinc (100-200 mg/L v. 1 mg/L in plasma) [Owen et al, J. Androl., 26, pages 459-469, 2005]. Zinc (like AP and PSA) is produced by the prostate gland and after ejaculation, 50% is bound to seminal vesicle proteins. Zinc acts to stabilize DNA inside spermatozoa, is a cofactor in enzymatic reactions and also may catalyze the gel-forming reaction between semenogelin I and II. Semen may be detected by the modified zinc test of Hooft and van de Voorde [Hooft et al, Forensic Sci. Int., 53, pages 131-133, 1992], which forms the principle of the zinc test in the present invention The semen flowing back out of a woman's vagina (“backflow”) is deposited on her underwear or absorbent pad. These items conveniently can be tested with the zinc test strip described in this invention. The strip also can be used to test stains on other fabrics and surfaces.

All stains on women's undergarments are not semen. In fact, asymptomatic women produce, on the average, 1.5 g of vaginal fluid per day, which typically leaves a white-to-beige stain [Beckmann et al. “Obstetrics and Gynecology, Second Edition”; Williams & Wilkins: Baltimore, page 294, 1995]. Semen stains, on the other hand are white and appear mainly just after intercourse. The next day, discharge of residual semen may not be visible at all. Thus, it is impossible to tell visually whether a suspicious stain is semen, and men must rely on analytical methods of detection such as that described in the present invention.

Other methods for semen detection have been described. Immunochromatographic test strips for PSA, first described by Yoshiki [An et al, Cancer Lett., 162, pages 135-9, 2001] are commercially available from several suppliers and have been validated for use in forensic investigations [Laux et al, online, retrieved 2008]. A similar test for semenogelin recently has been described [Pang et al, Forensic Sci. Int., 169, pages 27-31, 2007]. Test methods for acid phosphatase (AP) also have been described, for example as a test strip in U.S. Pat. No. 5,981,206 (Arter et al) and as a solution in U.S. Pat. No. 3,002,893 (Babson) and U.S. Pat. No. 6,764,856 (Holmes et al). Machery-Nagel also manufactures a test paper for the determination of AP, but they do not disclose the chemistry employed [Machery-Nagel, online, retrieved 2008].

Recently Hooft et al showed that the modified zinc test was more sensitive and specific for the detection of semen than the classic acid phosphatase test [Hooft et al, Am. J. Forensic Med. Pathol., 18, pages 45-49, 1997], although the ready availability of AP test strips may be a reason why zinc strips were not more widely adopted.

Although test methods for zinc have been described, a test strip assembly which allows for convenient handling and for the ready determination of semen has not.

### SUMMARY OF THE INVENTION

The present invention comprises an analytical test strip assembly and method for the detection of semen on garments and other items. The test strip consists primarily of a paper element coated with reagents which react to the presence of zinc, a component of semen, which element is affixed to a plastic backing to facilitate testing items without touching the highly sensitive coated portion of the strip. This assembly is exposed to a source of semen, such as a suspicious stain on a garment. A positive test is characterized by a bright pink color, which easily can be seen against the light yellow background. A positive test provides presumptive evidence of semen. This method provides for the convenient detection of semen when a suspect garment is tested less than 17 hours after intercourse.

The strip is designed to be easy to use and yields instant results. It is designed to detect traces of semen on a woman's undergarment which has been discharged after sexual intercourse, and up to 17 hours later.

The zinc strips can detect semen down to a 1/250 dilution. They are designed to be used by men who suspect their spouse may be engaged in sexual activity outside of their relationship. It also can be used by professional investigators, and parents concerned about whether their teenage daughters are sexually active.

The preferred method for the detection of semen on fabrics is to wet the suspect area with a few drops of water, and then press a zinc strip against it. A color change within 60 seconds to bright pink is a POSITIVE test.

### DETAILED DESCRIPTION OF THE INVENTION

The present invention relates to an analytical test strip and method for the identification of semen in suspicious stains on garments and other items. The strip is comprised of a paper element which reacts to the presence of semen, and is affixed to a plastic backing. The strips are sensitive to air, light and moisture and typically are packaged in a tightly sealed canister with a desiccant cap.

The preferred method for analyzing fabrics is to wet the suspect area with 5-10 drops of water, and then press a zinc strip against it. A color change within 60 seconds to bright pink is a POSITIVE test. A specific example of the preferred method is given below:

### Simple Procedure

1. Zinc test. Place 5-10 drops of distilled or deionized water on a suspect area of the garment. Press a zinc strip against it. A color change within 60 seconds from yellow to bright pink is a POSITIVE test.
Alternative procedure: place a wet cotton-tipped swab against the wetted area of the garment, wrap the garment around the swab and then press the cotton-tipped swab against a zinc test strip. This procedure yields a high-contrast pink spot against a yellow background, and will avoid leaving any stains on the garment.
NOTE: latex gloves are recommended for these procedures.

The zinc test strip assemblies are prepared according to a modification of the method of Hooft et al [Hooft et al, Forensic Sci. Int., 53, pages 131-133, 1992]. In the preferred embodiment, Whatman Grade 3 Qualitative Standard Filter Paper is used to absorb an emulsion of 1-(2-pyridylazo)-2-naphthol (PAN) according to the procedure of Hooft et al, then dried and cut into strips of approximately 16 mm by 41 mm. The strips then are mounted to a vinyl backing with adhesive. The preferred method consists of holding the assembly by the plastic portion and using it to carry out a detection test for semen as described above. This scheme allows users to handle the test strips without touching the highly sensitive coated portion of the strip. The strips are extremely sensitive to even the smallest trace of zinc (for example, from a user's finger), and it is not intuitively obvious that mounting the strips, prepared according to the literature procedure, as an assembly would facilitate the detection of zinc by the preferred method. The dimension of the final strip assembly is typically 16 mm by 60 mm. The strips are validated using standard solutions of zinc chloride, by which method their sensitivity can be determined. In a typical batch, the strips are found to have a detection limit of 1 mg of zinc per liter of water. When the strips are tested against a series of semen dilutions, they typically can detect semen down to a dilution of 1/250. Since semen typically has a zinc concentration of 100-200 mg/L [Owen et al, J.Androl., 26, pages 459-469, 2005], the two test methods are in general agreement with each other.

By means of the preferred method, the zinc strips typically can detect semen on women's undergarments which has been discharged up to 17 hours after intercourse. Garments tested closer to the time of intercourse give a more strongly POSITIVE spot test. The intensity of the spot test generally decreases linearly with time, but more rapidly after 12 hours. This observation can be attributed to secretion of zinc from the vagina along with normal vaginal fluid, rather than denaturization which is the case with seminal marker proteins.

While the invention has been described in detail with respect to the preferred embodiments thereof, it must be understood that changes can be made within the spirit and scope of the invention. All references cited herein, including patents, books, journal articles and other published prior art are incorporated for the purpose of teaching and understanding pertinent to this invention.

I claim:

1. A test strip assembly for the detection of semen, consisting of a coated paper element which reacts to the presence of zinc, a component of semen, which element is affixed to a plastic backing.

2. A method for the detection of semen using the test strip assembly in claim 1.
